(19) United States
(12) Patent Application Publication
(10) Pub. No.: US 2010/0292193 A1
(43) Pub. Date: Nov. 18, 2010

(54) RADIOPROTECTIVE DRUGS

(75) Inventors: WILLIAM H. McBRIDE, Los Angeles, CA (US); KWANGHEE KIM, Los Angeles, CA (US); ROBERT D. DAMOISEAUX, Beverly Hills, CA (US); ANDREW J. NORRIS, Los Angeles, CA (US)

Correspondence Address: Weaver Austin Villeneuve & Sampson LLP, P.O. BOX 70250, OAKLAND, CA, 94612-0250, US

(73) Assignee: THE REGENTS OF THE UNIVERSITY OF CALIFORNIA, Oakland, CA (US)

(21) Appl. No.: 12/757,786

(22) Filed: Apr. 9, 2010

Related U.S. Application Data
(60) Provisional application No. 61/168,541, filed on Apr. 10, 2009.

(51) Int. Cl. A61K 31/407; A61K 31/496; A61K 31/65; A61K 31/4709; A61P 39/00; A61P 35/00; A61P 31/00

(52) U.S. Cl. 514/152; 514/410; 514/253.08; 514/300

(57) ABSTRACT

Drugs and their compositions useful in preventing and treating negative side effects associated with radiation exposure or clinical radiotherapy are disclosed. More specifically, new compounds that can be administered systemically to patients exposed to radiation or undergoing radiotherapy and methods of using these formulations are disclosed.

### CROSS-REFERENCE TO RELATED APPLICATIONS

This application claims benefit of and priority to U.S. Ser. No. 61/168,541, filed on Apr. 10, 2009, which is incorporated herein by reference in its entirety for all purposes.

### STATEMENT OF GOVERNMENTAL SUPPORT

This invention was made with Government support of Grant No. AI067769 awarded by the National Institutes of Health. The Government has certain rights in this invention.

### FIELD OF THE INVENTION

This invention relates to the development of novel drugs to reduce or mitigate the effect of radiation on mammalian cells. More specifically, the present invention provides chemical compounds and their derivatives that can reduce or prevent the negative effects from radiation exposure from both clinical and non clinical sources.

### BACKGROUND OF THE INVENTION

It is generally accepted that DNA a primary target in the cytotoxic effects of ionizing radiation. The DNA damage results from both direct ionization in the DNA molecule (direct effect) and by indirect effects mediated by the radiolysis products of water. There is considerable evidence to support the view that DNA double-stranded (ds) breaks are particularly important. Ionizing radiation also induces damage in DNA bases. If the level of cellular DNA damage is sufficient, the consequence of irradiation is cell killing, and thus ionizing radiation is used as a mode of cancer therapy.

For humans and other animals, hematopoietic tissues and hematopoiesis are the most radiosensitive organs and function, followed by the gastrointestinal and other mucosa. Hematopoietic complications of radiation exposure (e.g., radiotherapy) can include, but are not limited to fatigue, petechial hemorrhages in the skin, ulceration of the mouth, epilation, anemia, and infections. Gastrointestinal complications of radiation exposure include nausea, vomiting, and prolonged diarrhea. Skin complications can include fibrosis, dry desquamation and moist desquamation. Mucosal complications in the eyes, nose, mouth, vagina, rectal mucosa and the like include dry mouth, difficulty swallowing, and mucositis that can lead to ulceration. Such conditions can result in an inability to tolerate food or fluids or limit the patient's ability to tolerate further radiotherapy or chemotherapy.

Finally, even if the radiation induced damage is sublethal, long term damage to soft tissues, such as fibrosis, and to the central nervous system, such as neurological symptoms and blindness, can be very debilitating. In addition, mutagenic lesions can have serious long term consequences, including carcinogenesis.

Medical strategies or countermeasures aimed at reducing the extent of the above radiation-induced effects are broadly described as radioprotectors. Radioprotectors include those agents that are effective when administered prior to radiation exposure, as well as agents that are effective if administered after irradiation, but before the appearance of symptoms, and agents that are effective if administered after the appearance of symptoms, which may mitigate symptoms or may treat established complications.

The commercial potential of radioprotectors resides primarily in two distinct arenas. One of these relates to the need to protect normal tissues in cancer radiotherapy patients or mitigate or treat normal tissue complications, and the other concerns the need to assuage the consequences of unplanned irradiation associated with civil scenarios, such as radiation accidents and radiation terrorism, as well as irradiation in military contexts

There are also no systemically used drugs that are fully approved by the U.S. Food and Drug Administration (FDA) for human use in nonclinical settings for the purpose of providing radiation protection to the public (Seed (2005) Health Phys, 89(5): 531-451).

Despite the absence of approved radioprotectors for nonclinical use, the global increase in the use and storage of radioactivity is increasing rapidly. Millions of radioactive sealed sources are used around the world for legitimate and beneficial commercial applications such as cancer treatment, food and blood sterilization, oil exploration, remote electricity generation, radiography, and scientific research. These applications use isotopes such as Cesium-137, Cobalt-60, Strontium-90, Americium-241, Iridium-192, Plutonium-238, Plutonium-239, Curium-244, Radium-226, and Californium-252. Furthermore, many of these radiological sources at sites around the world are no longer needed and have been abandoned or orphaned; others are poorly guarded, making the risk of theft or sabotage significant. Currently, there are tens of thousands of civilian locations worldwide containing radioactive material, about 5,000 of which contain radiation sources of 1,000 curies or greater (Office of Global Threat Reduction (NA-21). GTRI Strategic Plan, release date January 2007. 955 L'Enfant Plaza, Washington, D.C. 20585; Iliopulos et al. (2007) JNMM 35(3): 36-40).

Beyond the public safety concerns radioprotectors are of value in the clinical setting. About half of all cancer patients receive some type of radiation therapy and many receive multiple forms of radiation when treated. The number of cancer cases in the United States alone is over 1,400,000 (American Cancer Society, 2009) which would amount to more than 700,000 individuals exposed to therapeutic doses of radiation on an annual basis. Clinical radiation sources include beam sources (e.g., X-ray, gamma rays, proton beams, etc.) and material sources (e.g., as radium, uranium, cesium 131, cobalt 60, samarium 145, iodine 125 and 127, etc.) that for example may be applied on and/or around a tumor site, or systemically, parenterally, or orally administered.

### SUMMARY OF THE INVENTION

In various embodiments this invention pertains to the identification of a number of radioprotective agents (radioprotectors) that are useful in clinical and non-clinical contexts. In certain embodiments the radioprotectors comprise cyclopiazonic acid (CPA), a cyclopiazonic acid derivative and/or certain tetracycline derivatives.

Accordingly, in certain embodiments, methods are provided for protecting a cells, tissues, or organ(s) in a subject from radiation damage, or reducing radiation damage to cells, tissues, or organ(s) in a subject. The methods typically involve administering to the subject cyclopiazonic acid (CPA) and/or one or more cyclopiazonic acid derivative(s) and/or a tetracycline derivative in an amount effective to reduce radiation damage in a cell, tissue, or organ in said subject. In certain embodiments the cyclopiazonic acid (CPA) and/or a cyclopiazonic acid derivative comprises cyclopiazonic acid (see, e.g., Formula I or FIG. 1, or a pharmacologically acceptable salt, ester, or solvate thereof.

In certain embodiments the cyclopiazonic acid (CPA) and/or a cyclopiazonic acid derivative comprises a compound according to Formula II:

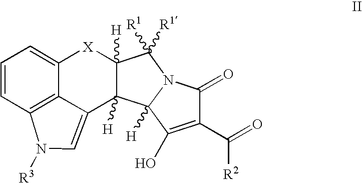

where X is selected from the group consisting of CH₂, O, NH, C₂H₄ and S; R¹ and R^{1′} are independently selected from the group consisting of H, F, Cl, CH₃, CH₂OH, and NH₂; R² is selected from the group consisting CH₃, (CH₂)ₙCH₃ where n=1, 2, 3 or 4, OH, (CH₂)ₙOH where n=1, 2, 3 or 4, NH₂, ester linked and ether linked alkyl group of the formula (CH₂)ₙCH₃ where n is between 0 and 24 and contains 0, 1, 2, 3 double bonds and 0, 1, 2, or 3 hydroxy moieties and one or two carbonyl moieties; and R³ is selected from the group consisting of H, methyl, ethyl, propyl, iso-propyl, butyl, iso-butyl, CF₃, CCl₃, benzyl and substituted benzyl derivatives, anthranyl and substituted derivatives, tosyl/sulfonamide, and an amino acid. In certain embodiments R¹ and R^{1′} respectively are selected from the group pairs of groups shown in Table 1 herein, e.g., H and H, H and Cl, H and F, F and F, CH₃ and H, CH₂OH and H, NH₂ and H, and CH₂OH and CH₃; or a salt, solvate, or ester thereof. In certain embodiments R² comprises a moiety selected from the group consisting of a CH₂, a CH₃, an H, an OH, a hemisuccinate, a choline, a phosphate, a phosphoryloxymethylcarbonyl, an amino acid, a dimethylaminoacetate, a phosphonate, an N-alkoxycarbonyl, and a phosphoryloxymethyloxycarbonyl. In certain embodiments R² and/or R³ comprise an amino acid selected from the group consisting of alanine, arginine, asparagine, aspartic acid, cysteine, glutamine, glutamic acid, glycine, histidine, isoleucine, leucine, isoleucine, lysine, methionine, phenylalanine, proline, pyrrolysine, serine, selenocysteine, threonine, tryptophan, tyrosine, and valine.

In certain embodiments the cyclopiazonic acid (CPA) and/or a cyclopiazonic acid derivative comprises a compound according to Formula III:

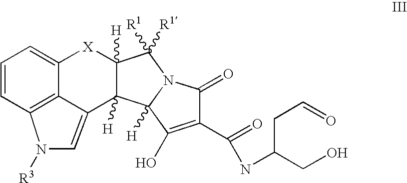

where X, R¹, R¹′, and R³ are as defined above. In certain embodiments X is N, CH₂, S, or C₂H₄. In certain embodiments R¹ and R¹′ respectively are selected from the group consisting of H and H, H and Cl, H and F, F and F, CH₃ and H, CH₂OH and H, NH₂ and H, and CH₂OH and CH₃ (e.g., as shown in Table 1 herein). In certain embodiments R³ is H.

In certain embodiments the cyclopiazonic acid (CPA) and/or a cyclopiazonic acid derivative comprises a compound according to Formula IV:

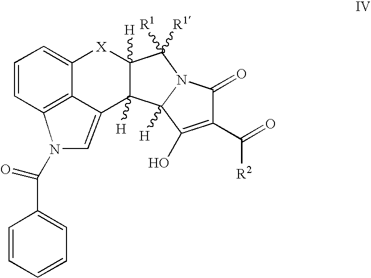

where X, R¹, R¹′ and R² are as defined above. In certain embodiments X is CH₂, C₂H₄, N, or S. In certain embodiments R¹ and R¹′ respectively are selected from the group consisting of H and H, H and Cl, H and F, F and F, CH₃ and H, CH₂OH and H, NH₂ and H, and CH₂OH and CH₃ (e.g., as shown in Table 1 herein). In certain embodiments R² is H, OH, CH₃, or one of the moieties listed in Table 2.

In various embodiments the cyclopiazonic acid (CPA) and/or a cyclopiazonic acid derivative is administered before, and/or during, and/or after exposure of said subject to radiation. In various embodiments the cyclopiazonic acid (CPA) and/or a cyclopiazonic acid derivative is combined with a pharmaceutically acceptable excipient or carrier. In certain embodiments the excipient or carrier is formulated to provide sustained release of the cyclopiazonic acid (CPA) and/or a cyclopiazonic acid derivative for a period of at least 2 hours, preferably at least 4 hours or at least 8 hours, more preferably at least 12 hours, 24 hours, 48 hours, and most preferably at least 3 days, at least 4 days, at least 5 days, at least one week, at least two weeks, or at least one month. In certain embodiments the excipient or carrier is formulated for administration via a route selected from the group consisting of oral administration, inhalation, rectal administration, surgical implantation, transdermal administration, parenteral administration, intravenous administration, subcutaneous administration, and topical administration. In certain embodiments the cyclopiazonic acid (CPA) and/or a cyclopiazonic acid derivative is administered via a route selected from the group consisting of oral administration, inhalation, rectal administration, surgical implantation, transdermal administration, parenteral administration, intravenous administration, subcutaneous administration, and topical administration. In certain embodiments the cells, tissues, or organs comprise a hematopoietic tissue or a mucosal tissue. In certain embodiments the subject is a non-human mammal (e.g., canine, bovine, porcine, feline, lagomorph, equine, non-human primate, etc.), or a human. In certain embodiments the radiation is produced in a therapeutic treatment (e.g., by an implanted radiation source, by a beam radiation source, etc.). In certain embodiments the cyclopiazonic acid (CPA) and/or a cyclopiazonic acid derivative is administered in conjunction with an anti-cancer drug. In certain embodiments the radiation is produced in a non-clinical setting.

In certain embodiments methods of cancer radiotherapy or radiosurgery are provided. The methods comprise administering to non-tumor cells and/or tissues and/or organs in a subject in need of such therapy an amount of a cyclopiazonic acid (CPA) and/or a cyclopiazonic acid derivative, and/or tetracycline, and/or a tetracyclinederivative effective to reduce radiation damage to the non-tumor cells and/or tissues, and/or organs; and subjecting a tumor or a metastatic cell in the subject to radiation. In certain embodiments the tumor or metastatic cell to be treated is of a cancer selected from the group consisting of lung cancer, colorectal cancer, NSCLC, bronchoalveolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis. In certain embodiments the tumor or tumor metastasis is refractory. In various embodiments the cyclopiazonic acid, cyclopiazonic acid derivative comprises cyclopiazonic and/or one or more of the cyclopiazonic acid derivatives described herein (e.g., compounds according to Formulas I, II, III, or IV as described herein). In various embodiments the cyclopiazonic acid (CPA) and/or a cyclopiazonic acid derivative is administered before and/or during and/or after exposure of the subject to radiation. In certain embodiments the cyclopiazonic acid (CPA) and/or a cyclopiazonic acid derivative is combined with a pharmaceutically acceptable excipient or carrier. In certain embodiments the excipient or carrier is formulated to provide sustained release of the cyclopiazonic acid (CPA) and/or a cyclopiazonic acid derivative as described above. In certain embodiments the excipient or carrier is formulated for administration via a route selected from the group consisting of oral administration, inhalation, rectal administration, surgical implantation, transdermal administration, subcutaneous administration, parenteral administration, subcutaneous administration, intravenous administration, and topical administration. In certain embodiments the cyclopiazonic acid (CPA) and/or a cyclopiazonic acid derivative is administered via a route selected from the group consisting of oral administration, inhalation, rectal administration, surgical implantation, transdermal administration, parenteral administration, intravenous administration, subcutaneous administration, and topical administration. In certain embodiments the cells, tissues, or organs comprise a hematopoietic tissue or a mucosal tissue. In various embodiments the subject is a non-human mammal, or a human. In certain embodiments the radiation is produced by an implanted radiation source and/or by a beam radiation source. In certain embodiments the cyclopiazonic acid (CPA) and/or a cyclopiazonic acid derivative is administered in conjunction with an anti-cancer drug.

Also provided are methods of protecting biological material from radiation damage, or reducing radiation damage in biological material. The methods typically involve exposing the biological material to a cyclopiazonic acid (CPA) and/or a cyclopiazonic acid derivative in an amount sufficient to reduce or inhibit damage from exposure to radiation. In certain embodiments radiation is from a clinical radiation source. In certain embodiments the radiation is from a non-clinical radiation source. In various embodiments the cyclopiazonic acid (CPA) and/or a cyclopiazonic acid derivative is a cyclapiazonic acid or derivative thereof according to Formulas I, II, III, or IV as described herein.

Pharmaceutical compositions are also provided. In various embodiments the compositions comprise cyclopiazonic acid (CPA) and/or a cyclopiazonic acid derivative in a pharmaceutically acceptable excipient or carrier. In various embodiments the cyclopiazonic acid (CPA) and/or a cyclopiazonic acid derivative comprises a compound according to Formula I, II, III, or IV as described herein. In various embodiments the excipient or carrier is for administration in a modality suitable for inhibiting cell or tissue damage from radiation exposure. In certain embodiments the composition additionally comprises one or more other anti-cancer agents. In certain embodiments the other anti-cancer agent is selected from the group consisting of an alkylating drug, an antimetabolite, a microtubule inhibitor, a podophyllotoxin, an antibiotic, a nitrosourea, a hormone, a kinase inhibitor, an activator of tumor cell apoptosis, and an antiangiogenic agent.

In certain embodiments a pharmaceutical composition for oral administration to a mammalian subject is provided. In certain embodiments the composition comprises cyclopiazonic acid (CPA) and/or a cyclopiazonic acid derivative as described herein (e.g., a compound according to Formula I, II, III, or IV as described herein), and a vehicle comprising i) a TWEEN surfactant at ranging from 0.01% to about 10% by volume in a biologically compatible solvent; and ii) a carrier comprising at least 1-30% Vitamin E TPGS. In certain embodiments the biologically compatible solvent is selected from the group consisting of sterile water, PBS and normal saline. In certain embodiments the composition further comprises ethanol, polyethylene glycol, and/or propylene glycol.

In various embodiments methods are provided for treating tumors or tumor metastases in a patient. The methods involve administering to the patient a therapeutically effective amount of a pharmaceutical composition comprising at least one cyclopiazonic acid (CPA) and/or a cyclopiazonic acid derivative as described herein in pharmaceutically acceptable excipient, carrier or vehicle. In certain embodiments the patient is a human that is being treated for cancer, in preventiye and/or active disease situations. In certain embodiments the tumor or tumor metastases to be treated is selected from the group consisting of lung cancer, colorectal cancer, NSCLC, bronchoalveolar cell lung cancer, bone cancer, pancreatic cancer, skin cancer, cancer of the head or neck, cutaneous melanoma, intraocular melanoma, uterine cancer; ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastases. In certain embodiments the tumors or tumor metastases are refractory. In certain embodiments the tumors or tumor metastases to be treated are NSCLC tumors or tumor metastases. In various embodiments the method additionally comprises administering one or more other anti-cancer agents. In certain embodiments the cyclopiazonic acid and/or cyclopiazonic acid derivative is administered via a route selected from the group consisting of oral administration, inhalation, rectal administration, surgical implantation, transdermal administration, parenteral administration, intravenous administration, subcutaneous administration, and topical administration. In certain embodiments the composition is administered to prevent and/or treat non-cancer diseases or conditions that result from changes in cellular proliferation selected from benign hypertrophy of tissues, arthritis, retinal ailments, skin abnormalities, scar formation, cardiovascular diseases, gastrointestinal dysfunction, hematologic illness, immunological imbalance, allergies, gynecological and urological problems. In certain embodiments the composition is administered to prevent and/or treat non-cancer diseases or conditions that result from changes in angiogenesis process selected from ailments/conditions that result from too high or too low levels of blood vessel formation. In certain embodiments the composition is administered to treat one or more infections caused by one or multiple agents selected from bacteria, fungi, viruses, mycobacteria, and yeast as a consequence of radiation exposure.

In various embodiments methods are provided for protecting a cell, and/or a tissue, and/or an organ in a subject from radiation damage, or reducing radiation damage to cells or tissues in a subject, the method comprising administering to the subject an agent selected from the group consisting norfloxacin, meclocycline, and moxifloxacin in an amount effective to reduce radiation damage in a cell, tissue, or organ in the subject. In various embodiments the subject is a human or a non-human mammal. In various embodiments the subject is exposed to radiation treatment.

In certain embodiments, the methods and formulations described herein expressly exclude one or more agents selected from the group consisting of tetracycline, oxytetracycline, cholorotetracycline, doxycycline, ascorbate, quinolone derivatives, ceftriaxone, and dipyridamole.

### DEFINITIONS

As used herein, the term “cancer” in a mammal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

As used herein, the term “therapeutically effective amount” or “effective amount” means an amount sufficient to effect beneficial or desired results. An effective amount can be administered in one or more administrations.

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs).

As used herein, the term “derivative” of a compound refers to a chemically modified compound wherein the chemical modification takes place either at a functional group of the compound, aromatic ring, or carbon backbone; including, for example, esters of alcohol-containing compounds, esters of carboxyl-containing compounds, amides of amine-containing compounds, amides of carboxyl-containing compounds, imines of amino-containing compounds, and the like.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases well known to those skilled in the art.

As used herein, the term “administration” refers to the act of giving a drug, prodrug, or other agent, or therapeutic treatment (e.g., radiation therapy) to a physiological system (e.g., a subject or in vivo, in vitro, or ex vivo cells, tissues, and organs). Illustrative routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, into cerebrospinal fluid, etc.) and the like.

In this specification “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, alkynyloxy, aryloxy, carboxy, benzyloxy haloalkoxy, haloalkenyloxy, haloalkynyloxy, haloaryloxy, nitro, nitroalkyl, nitroalkenyl, nitroalkynyl, nitroaryl, nitroheterocyclyl, azido, amino, alkylamino, alkenylamino, alkynylamino, arylamino, benzylamino, acyl, alkenylacyl, alkynylacyl, arylacyl, acylamino, acyloxy, aldehydro, alkylsulphonyl, arylsulphonyl, alkylsulphonylamino, arylsulphonylamino, alkylsulphonyloxy, arylsulphonyloxy, heterocyclyl, heterocycloxy, heterocyclylamino, haloheterocyclyl, alkylsulphenyl, arylsulphenyl, carboalkoxy, carboaryloxy, mercapto, alkylthio, arylthio, acylthio and the like.

The salts of the compounds of Formulas I and II are in certain embodiments, pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicyclic, sulphanilic, aspartic, glutamic, edetic, stearic, palmitic, oleic, lauric, pantothenic, tannic, ascorbic and valeric acids.

By “pharmaceutically acceptable derivative” is meant any pharmaceutically acceptable salt, hydrate, solvate or any other compound which, upon administration to the subject, is capable of providing (directly or indirectly) a compound of Formula I, Formula II, another radioprotective agent described herein, and/or an active metabolite or residue thereof.

The term “pro-drug” is used herein in its broadest sense to include those compounds which are converted in vivo to compounds of Formula I, Formula II, or other radioprotective agents described herein.

### BRIEF DESCRIPTION OF THE DRAWINGS

FIG. 1 illustrates a structure for a Cyclopiazonic acid (CPA).

FIG. 2 illustrates other radioprotective agents.

FIGS. 3A and 3B show radiation dose-responses of irradiated TiL1 cells treated with cyclopiazonic acid (CPA). Cell viability assay with TiL1 cells was measured using ATPlite reagent 24 h after irradiation with 2Gy. CPA was added to the cells 3 h before IR for protection (FIG. 3A) or 1 h after IR for mitigation (FIG. 3B) activities.

FIGS. 4A and 4B show the effect of CPA on animal survival against a lethal dose total body irradiation (TBI). FIG. 4A: Two oral administrations of CPA at 24 h and 1 h prior to irradiation at 8 Gy. FIG. 4B: CPA at 6 mg/kg or vehicle control was administered twice prior to irradiation as (FIG. 4A) along with un-irradiated control mice for Granulocyte-macrophage colony forming units.

FIGS. 5A and 5B show the effect of CPA on ROS scavenging. CPA did not reduce the irradiation induced reactive oxygen species (FIG. 5A), while di-tBHQ did in dose-responsive manner (FIG. 5B).

### DETAILED DESCRIPTION

In various embodiments this invention pertains to the identification of radioprotective compounds (agents) and to uses thereof. The radioprotective compounds are useful as radiotherapeutic compounds to prevent, mitigate, or treat radiation induced damage to cells tissues, or organs, and/or organisms, that have already been exposed to radiation (e.g., from clinical or non-clinical sources), or as prophylactics to mitigate or prevent damage to cells tissues or organs, and/or organisms that are expected to be exposed to radiation (e.g., in anticipation of radiotherapy, in certain military contexts, and the like).

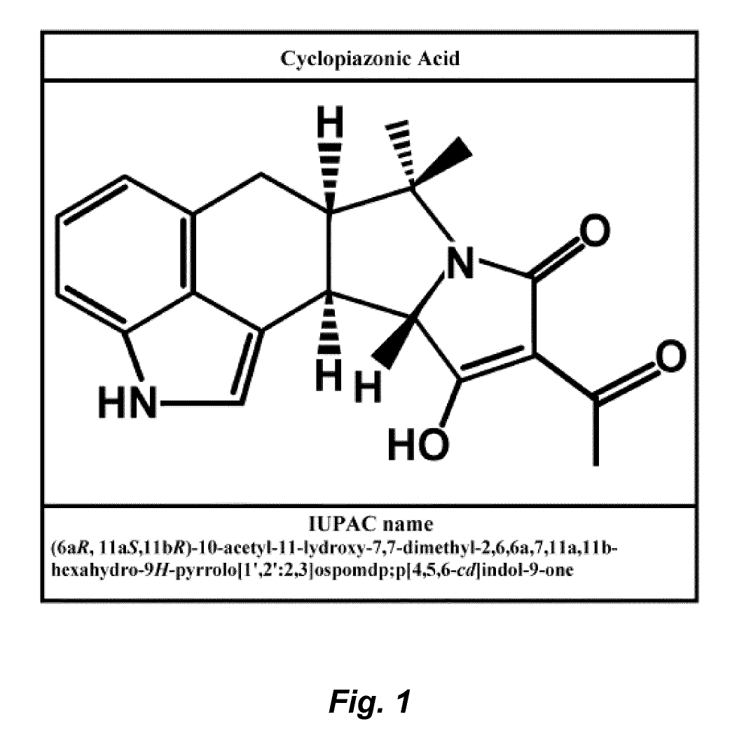

Also provided are methods of protecting a subject or biological material from radiation damage, or of reducing radiation damage to a subject or biological material. The methods involve administering to the subject, or exposing the biological material to, an effective amount of one or more radioprotector compound(s) described herein (e.g., cyclopiazonic acid (CPA) (see, e.g., FIG. 1, Formula I), a cyclopiazonic acid derivative, and/or another radioprotective agent described herein). In certain embodiments the cyclopiazonic acid (CPA) is a cyclopiazonic acid according to Formula I shown below and/or the cyclopiazonic acid derivative is a derivative in accordance with Formula II shown below.

By the phrase protecting from radiation damage it is implied that relative to damage expected to be incurred to cells, tissue, or organism within a subject or within biological material following exposure to a given amount of radiation (for example ionizing, infra-red or ultra-violet radiation) damage is prevented, minimized or reduced due to effect of the radioprotector compound.

The radiation damage may result from exposure to a radiation source, such as, ionizing radiation. The term “ionizing radiation” as used herein refers to photons having enough energy to ionize a bond, such as, alpha, beta, and gamma rays from radioactive nuclei and x-rays.

The term “biological material” is used herein in its broadest sense and includes any composition of matter which comprises at least one biologically-derived or derivable component. Biological material contemplated by the present invention includes proteins and other proteinaceous material including extracts of or including proteins and chemically modified proteins or extracts thereof; tissue fluids, tissue extracts or organs; animal, plant or microbiological tissue, fluid or extracts including products therefrom; biologically derived non-proteinaceous material such as, but not limited to, lipids, carbohydrates, hormones and vitamins including extracts and derivatives thereof; recombinant products including genetic material such as chromosomal material, genomic DNA, cDNA, mRNA, tRNA, ribosomes and nuclear material; and whole animal, plant or microbiological cells or extracts thereof.

As indicated the biological material of the invention can take the form of cells, tissues or organs or indeed of peptides, proteins or nucleic acids (for example) derived from a plant, animal or microorganism source, as well as those synthetically produced which mimic or are similar to naturally derived materials. The radioprotector compound can be used to protect from radiation damage for example in experimental systems, in whole live or dead organisms or on ex vivo cells, tissues or organs that may be returned to the original host, or transplanted into a new host, after therapy.

For example, the biological material can take the form of a human or animal subject such as an experimental animal (e.g., mouse, rat, guinea pig, rabbit), a companion animal (e.g., cat, dog), an agricultural animal (e.g., horse, cattle, sheep, donkey, goat, pig), a reptile, avian or captive wild animal. Preferably the subject is a mammal and most preferably the subject is a human.

A significant application for the radioprotector compounds described herein is for use in conjunction with radiotherapy in human or non-human subjects. However, the compounds can also be used to offer protection from exposure to, or from continuing exposure to, unplanned radiation such as in a terrorism, military or occupational context.

In certain embodiments the biological material (including the human or animal subject) is exposed to the radioprotector compound(s) for a sufficient period of time in advance of anticipated radiation exposure or continuing radiation exposure, such as between about 1 minute and about 3 days, preferably between about 10 minutes and about 6 hours, more preferably between about 20 minutes and about 4 hours and most preferably between about 30 minutes and about 2 hours.

In certain embodiments the radioprotector compound(s) are administered preferentially to cells, tissues or organs likely to be exposed to radiation but that are intended to be protected from such radiation exposure. For example, in the case of administration in conjunction with cancer radiotherapy the compounds will preferably be administered preferentially to normal (non-tumor) tissues or cells surrounding a tumor or lesion that are likely to be exposed to radiation in the course of radiotherapy. Preferential administration can be achieved by way of direct application to the desired cells or, for example, by utilizing a system for targeting specific cells or tissues. For example it is possible to conjugate the compounds to agents that preferentially bind to specific cells or tissues, such as to receptors that are up-regulated in the particular cells or tissues concerned.

In certain embodiments the radioprotective agents described herein can be conjugated to agents, for example, via an interactive group, that will specifically deliver them to a desired tissue or organ. Suitable agents may include antibodies or proteins, such as, growth factors, for example, haemopoietic growth factor which will enable preferential radioprotection of haemopoietic stem cells to occur in the context of total body irradiation and bone marrow transplantation. The term “interactive group” is used herein in its broadest sense and refers to a group capable of forming a bond with a specific group on a target molecule or agent such as a protein or a derivative thereof. Examples of interactive groups include, but are not limited to N(CH₂)ₙCOOH, N(CH₂)ₙCO(CH₂)ₙR, N(CH₂)ₙ—SH, N(CH₂)ₙ—NH₂, CH(CH₂)ₙCOOH, CH(CH₂)ₙCO(CH₂)ₙR, CH(CH₂)ₙ—SH and CH(CH₂)ₙ—NH₂ wherein n is 1 to 10, m is 0 to 10 and R is optionally substituted alkyl.

In certain embodiments, methods are provided for cancer radiotherapy that involve comprises administering to a subject in need of such therapy an effective amount of one or more radioprotector agent(s) described herein and subjecting the locus of the tumor to a radiation source. The term “cancer radiotherapy” is used herein in its broadest sense and includes radiotherapy involving tumors or lesions, which may be either benign or malignant.

The radioprotective agents described herein can also be used advantageously in therapy in combination with other medicaments, such as chemotherapeutic agents, for example, radiomimetic agents that are cytotoxic agents that cells, tissues, and/or organs in a manner similar to ionizing radiation. Examples of radiomimetic agents include, but are not limited to bleomycin, doxorubicin, adriamycin, SFU, neocarcinostatin, alkylating agents and other agents that produce DNA adducts.

In various embodiments it is believed the radioprotectors described herein will offer at least partial protection from damage by some of these agents, in the same way as they protect against the effects of ionizing radiation. In particular, in certain instances there are circumstances where topical application to problem tissues could be advantageous. For example, oral mucositis is a problem side-effect for cytotoxic agents, such as, doxorubicin and administration of the radioprotective agents described herein as a mouth-wash before administration of the chemotherapeutic agent could ameliorate this side-effect without compromising the action of this agent on a tumour not located in the oral cavity. Similarly, the gastrointestinal tract could be protected by oral administration, the lungs by aerosol inhalation or the bladder by intravesical delivery, for example, via a catheter of the radioprotector. Hence certain methods contemplate the use of the radioprotective agent(s) described herein in conjunction with another medicament, such as, a radiomimetic agent.

In one embodiment, one or more of the radioprotective agents described herein is applied topically to the skin at the site of entry during radiation therapy to effect radioprotection of the skin surface.

The radioprotective agent(s) described herein can also be used in ex vivo applications. One such application is in the context of bone marrow transplantation. Bone marrow transplantation generally involves obtaining and storing bone marrow samples from a subject in anticipation of a deterioration of their condition. High dose chemotherapy is administered. This chemotherapy is such that it would normally be lethal due to the destruction of normal stem cells, but the subject is rescued by the administration of their own haemopoietic stem cells. The problem with this procedure is that the initial sample of stem cells is likely to be contaminated with tumor cells and various procedures are used therefore to purge the bone marrow preparations of the tumor cells. Radioprotectors, conjugated for example to a haemopoietic growth factor or alone, can be used in this context by being added to a suspension of bone marrow cells. The suspension may then be irradiated in the expectation that the normal bone marrow cells, but not the tumor cells, would be preferentially protected from the cell-killing effects of the radiation.

In certain embodiments, methods of preventing, treating tumors or tumor metastases in a patient are also provided. In certain embodiments the methods comprise administering to the patient a therapeutically effective amount of a pharmaceutical composition comprising at least one cyclopiazonic acid or cyclopiazonic derivative compound in pharmaceutically acceptable excipient, carrier or vehicle. In some embodiments, the present invention provides a method for reducing cellular proliferation comprising the step of exposing a cyclopiazonic acid or cyclopiazonic acid derivative compound to cells. In some embodiments, the cellular proliferation is associated with cancer. In some embodiments, the cells are located in vivo in a subject (e.g., a human). In some embodiments, the cancer is pancreatic cancer, breast cancer, colon cancer, lung cancer, skin cancer, brain cancer, cervical cancer, ovarian, stomach cancer, or prostate cancer.

In certain embodiments the methods are provided for using one or more of the active agents described herein for partially or fully preventing and/or treating non-cancer diseases or conditions that result from changes in cellular proliferation or angiogenesis process. These non-cancer conditions may include but are not limited to benign hypertrophy of tissues, arthritis, retinal ailments, skin abnormalities, scar formation, cardiovascular diseases, gastrointestinal dysfunction, hematologic illness, immunological imbalance, allergies, gynecological and urological problems, bacterial infections etc. Diseases involving the angiogenesis process include ailments/conditions that result from too high or too low levels of blood vessel formation.

The foregoing uses are illustrative and not liming. Using the teaching provided herein, other uses of the radioprotective agents described herein will be readily available to one of skill in the art.

### I. Cyclopiazonic Acid (CPA) and Cyclopiazonic Acid Derivative(s),

It was discovered that cyclopiazonic acid is a potent radioprotector effective both as a radioprotective mitigator and a radioprotective preventative. Accordingly, in certain embodiments, cyclopiazonic acid, salts (e.g., pharmaceutically acceptable salts) thereof and/or solvates thereof are contemplated. In certain embodiments, a cyclopiazonic acid according to Formula I is contemplated:

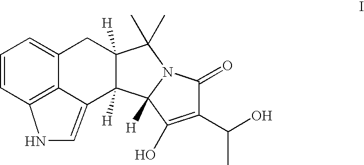

as are pharmacologically acceptable salts, and/or esters and/or solvates thereof. In addition, cyclopiazonic acid derivatives also having radioprotective activity are contemplated. In certain embodiments, a cyclopiazonic acid derivative according to Formula II is contemplated:

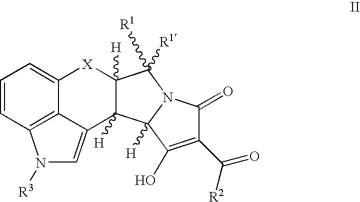

where X is selected from the group consisting of C, O, NH and S; R¹ and R^{1′} are independently selected from the group consisting of H, F, Cl, CH₃, CH₂OH, NH₂. R² is selected from the group consisting CH₃, (CH₂)ₙCH₃, where n=1, 2, 3 or 4; OH; (CH₂)ₙOH where n=1, 2, 3 or 4; NH₂; ester linked and ether linked alkyl group of the formula (CH₂)ₙCH₃ where n is between 0 and 24 and contains 0, 1, 2, 3 double bonds and 0, 1, 2, or 3 hydroxy moieties and one or two carbonyl moieties; and R³ is selected from the group consisting of H, methyl, ethyl, propyl, iso-propyl, butyl, iso-butyl, CF₃, CCl₃, benzyl and substituted benzyl derivatives, anthranyl and substituted derivatives, tosyl/sulfonamide, and an amino acid (naturally occurring or non-naturally occurring including D amino acids, norleucine, hydroxyproline, isovaline, and the like).
amino acids as below and un-natural amino acids such as D-alanine, norleucine etc,

In certain embodiments R² comprises a moiety selected from the group consisting of a hemisuccinate, a choline, a phosphate, a phosphoryloxymethylcarbonyl, an amino acid, a dimethylaminoacetate, a phosphonate, an N-alkoxycarbonyl, and a phosphoryloxymethyloxycarbonyl.

In certain embodiments, R² is selected from the group consisting of myristic acid, lauric acid, linoleic acid, oleic acid, levulinic acid (4-oxopentanoic acid), myristoleic acid, palmitoleic acid, sapienic acid, oleic acid, linoleic acid, α-linolenic acid, arachidonic acid, eicosapentaenoic acid, erucic acid, docosahexaenoic acid (see, e.g., Table 2 below).

In certain embodiments, R¹ and R¹′ are selected pairs as shown in Table 1.

TABLE 1
Illustrative combinations of R¹ and R¹′.

| | R¹ | R¹′ |
|---|---|---|
| 1 | H | H |
| 2 | H | Cl |
| 3 | H | F |
| 4 | F | F |
| 5 | CH₃ | H |
| 6 | CH₂OH | H |
| 7 | NH₂ | H |
| 8 | CH₂OH | CH₃ |

In certain embodiments, R¹ and R¹′ are as shown for species 1-8 in Table 1, and R² is selected from the species shown in Table 2.

TABLE 2
Illustrative moieties for R².

| | R² |
|---|---|
| 1 | linolenic acid |
| 2 | capric acid |
| 3 | myristic acid |
| 4 | lauric acid |
| 5 | linoleic acid |
| 6 | oleic acid |
| 7 | levulinic acid (4-oxopentanoic acid). |
| 8 | Myristoleic acid |
| | CH₃(CH₂)₃CH═CH(CH₂)₇COOH |
| 9 | Palmitoleic acid |
| | CH₃(CH₂)5CH═CH(CH₂)₇COOH |
| 10 | Sapienic acid |
| | CH₃(CH₂)8CH═CH(CH₂)₄COOH |
| 11 | Oleic acid |
| | CH₃(CH₂)7CH═CH(CH₂)₇COOH |
| 12 | Linoleic acid |
| | CH₃(CH₂)4CH═CHCH₂CH═CH(CH₂)₇COOH |
| 13 | α-Linolenic acid |
| | CH₃CH₂CH═CHCH₂CH═CHCH₂CH═CH(CH₂)₇COOH |
| 14 | Arachidonic acid |
| | CH₃(CH₂)4CH═CHCH₂CH═CHCH₂CH═CHCH₂CH═CH(CH₂)₃COOH |
| 15 | Eicosapentaenoic acid |
| | CH₃CH₂CH═CHCH₂CH═CHCH₂CH═CHCH₂CH═CHCH₂CH═CH(CH₂)₃COOH |
| 16 | Erucic acid |
| | CH₃(CH₂)7CH═CH(CH₂)₁₁COOH |
| 17 | Docosahexaenoic acid |
| | CH₃CH₂CH═CHCH₂CH═CHCH₂CH═CHCH₂CH═CHCH₂CH═CHCH₂CH═CH(CH₂)₂COOH |

In certain embodiments, R² and/or R³ is H and R¹ and R¹′ and R² are as described above. In certain embodiments R² and/or R³ is H and R¹ and R¹′ are as shown in Table 1.

In certain embodiments the cyclopiazonic acid derivative comprises a compound according to Formula III:

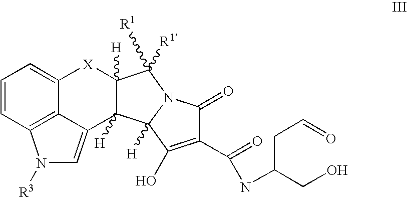

where X, R¹, R¹′, and R³ are as defined above. In certain embodiments X is N, CH₂, S, or C₂H₄. In certain embodiments R¹ and R¹′ respectively are selected from the group consisting of H and H, H and Cl, H and F, F and F, CH₃ and H, CH₂OH and H, NH₂ and H, and CH₂OH and CH₃ (e.g., as shown in Table 1 above). In certain embodiments R³ is H.

In certain embodiments the cyclopiazonic acid derivative comprises a compound according to Formula IV:

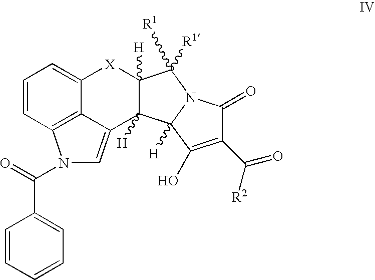

where X, R¹, R¹′ and R² are as defined above. In certain embodiments X is CH₂, C₂H₄, N, or S. In certain embodiments R¹ and R¹′ respectively are selected from the group consisting of H and H, H and Cl, H and F, F and F, CH₃ and H, CH₂OH and H, NH₂ and H, and CH₂OH and CH₃ (e.g., as shown in Table 1 above). In certain embodiments R² is H, OH, CH₃, or one of the moieties listed in Tablet.

In various embodiments salts and/or solvates, and/or esters of the compounds described above are contemplated. In addition, prodrug forms of a cyclopiazonic acid and/or cyclopiazonic acid derivatives are contemplated.

### Preparation of Cyclopiazonic Acid and Cyclopiazonic Acid Derivatives

Methods for the total chemical synthesis of cyclopiazonic acid and derivatives thereof are known to those of skill in the art (see, e.g., Kozikowski and Greco (1984) J. Chem. Soc., 106: 6873-6874 and references therein; Haskins and White (2005) Chem. Commun., 3162-3164 and references therein). Thus, for example, CPA can be produced in 11 steps from indole-4-methanol 6; the key step is a carbocationic cascade, terminated by a 4-nitrosulfonamide group and initiated by benzylic carbocation formation directly from the intermediate 9, which gives the tetracyclic product (see, Haskins and White supra). In addition, cyclopiazonic acid is commercially available (see, e.g., Sigma-Aldrich catalog).

In addition, cyclopiazonic acid and certain cyclopiazonic acid derivatives can be purified from various plants or fungi. Methods of purifying cyclopiazonic acid and certain cyclopiazonic acid derivatives from biological sources are known to those of skill in the art (see, e.g., Peterson et al. (1989) Assoc. Off. Anal. Chem., 72(2): 332-335). The method described by Peterson et al. crude cyclopiazonic acid was extracted from fermentation medium with chloroform-methanol (80+20), dried, dissolved in chloroform, and chromatographed on an oxalic acid/silica preparative column with chloroform-methanol (99+1) as the eluant. A semi-preparative oxalic acid/silica column and chloroform-methanol (99.5+0.5) were then used for rechromatography of the partially purified cyclopiazonic acid. This second chromatographic treatment yielded fractions from which cyclopiazonic acid was readily crystallized. Analytical chromatography was performed using an amino column in an ion-exchange mode, with a methanol-phosphate buffer eluant. Response was linear from 10 to 800˜g/injection of standard solutions. Cyclopiazonic acid chemically bound sodium from soda-lime vials. This purification method is illustrative and not limiting. Other purification methods are well known to those of skill in the art.

In certain embodiments of the active agent(s) are derived from plants or fungus. The fungus contemplated for use in the present invention can be any one of a wide variety of fungi such as Aspergillus flavus and the like.

The plants contemplated for use can include any one or more of a wide variety of plants and can include sexually or vegetatively propagated plants as further described herein. In particular, plants suitable for use in the invention, such as use in the method for eliciting a compound having therapeutic activity as described herein, include, for example: Livistona chinensis, Neptunia oleracea, Clerodendrum calamitosum, Clerodendrum cyrtophyllum, Atropa bella donna, Erythrina flabelliformis, Ipomoea tricolor, Erythrina crista, Celosia cristata, Gallium spurium, Laurus nobilis, Vitis labrusca, Vitis vinifera, Gratiola officinalis, Symphitum officinalis, Hosta fortunei, Cassia hebecaipa, Thalictrum flavum, Scutellaria altissima, Portulacca oleracea, Scutellaria certicola, Physalis sp., Geum fauriei, Gentiana tibetica, Linum hirsutum, Aconitum napellus, Podophyllum emodii, Thymus cretaceus, Carlina acaulis, Chamaecrista fasciculata, Pinus pinea, Peganumharmala, Tamarindus indica, Carica papaya, Cistus incanus, Capparis spinosa, Cupressus lusitanica, Diospyros kaki, Eryngium campestre, Aesculus woerlitzensis, Aesculus hippocastanum, Cupressus sempervirens, Celtis occidentalis, Polygonum cuspidatum, Elaeagnus angustifolia, Elaeagnus commutata, Gentiana macrophylla, Brassica rapa, Sesbania exaltata, Sesbania speciosa, Spartinapotentifiora, Brassica juncea, Helianthus annuus, Poinsettiasp., Pelargonium zonale, Synapsis sp., Leontopodium alpinum, Lupinus luteus, Buxus microphylla var. japonica, Liatris spicata, Primula japonica, Betula nigra, Filipendula vulgrais, Lobelia siphilitica, Grevillea robusta, Reseda luteola, Gentiana littoralia, Campanula carpatica, Ageratum conizoides, Psidium guajava, Ailanthus altissima, Hydrocotyle asiatica, Brugmansia suaveolens, Thymus pulegioides, Thymus lema-barona, Thymus serphyllum (wild), Gaultheria procumbens, Thymus camosus, Thymus thracicus, Calycanthus floridus, Zin giber officinalis, Lamium dulcis, Thymus praecox “arcticus”, Thymus speciosa, Thymus pseudolamginosus, Thymus vulgraris, Ficus religiosa, Forsythia suspensa, Chelidonium majus, Thymus wooly, Thymus portugalense, Nicotiana tabacum, Thymuscytriodorus “aureus”, Cactus officinailis, Lablab purpurea, Juglans regia, Actinidia chinensis, Hemerocallis sp., Betula pendula, Gardenia jasminoides, Taxodiumdistichum, Magnolia loebherii, Crataegus praegophyrum, Larix decidua, Thuja orientalis, Thuja ociden talis, Cupressocyparis leylandii, Pseudotsuga menziesii, Abiesfinna, Parthenocissus quinquefolia, Allium cemuum, Juniperus “blue pacific”, Taraxacum officinalis, Yucca sp., Tsuga canadensis, Ilex aquifolium, Ilex comuta, Taxus hiksii, Taxus media, Metasequoia glyptostroboides, Pinus bungi ana, Buxus sempervirens, Stewartia koreana, Prunus sp., Betula dahurica, Plantago minor Acer palmatum, Acer campestre, Cotinus coggygria, Quercus robur, Acer truncatum, Achyranthes bidentata, Allium japonicum, Carum cap sicum, Agastache mexicana, Prunella vulgaris, Tagetes rhinuta, Nepeta cataria, Ratibidacolumnaris, Aster novae angliae, Myrica cerifera, Pittosporum tobira, Plantago major, Pinus sylvestris, Acorus canadensis, Pieris japonica, Pinus strobus, Trifolium pratense, Prunus serotina, Datura stramonium, Geranium maculatum, Hydrocotyle asiatica, Astragalus sinicus, Centaurea maculata, Ruschia indurata, Myrthus communis, Platanus occidentalis, Licium barba turn, Lavandula officinalis, Grevillea robusta, Hypophae rhamnoides, Filipendula ulmaria, Betula pendula, Polygonum odoratum, Brugmansia graveolens, Rhus toxi codenta, Armoracia rusticana, Ficus benjaminii, Sufflera sp., Baikiaea recurvata, Asimina triloba, Lippia dulcis, Epilobium augustifolium, Brugmansia suaveolens, Xanthosoma sagittifolium, Monstera deliciosa, Aglaonema commutatus, Dieffenbachia leopoldii, Anthurium andreanum, Syngonium podophyllum, Dracaena fragrans, Ananas comosus, Strelitzia reginae, Dieffenbachia segiune, Syngonium auritum, Dracaena sp., Haemanthuskatharinae, Anthurium altersianum, Spathiphyllum grandifiorum, Spathiphyllum cochle arispatum, Monstera pertusa, Anthurium magnificum, Anthurium hookeri, Anthurium elegans, Calathea zebrina, Yucca elephantipes, Bromelia balansae, Musa textilis, Myrthus communis, Olea oleaster, Olea europaea, Nerium oleander, Cocculus laurifolius, Microsorium punctatum, Sanseviera sp., Adansonia digitata, Boehmeria biloba, Piper nigrum, Phymatosorus scolopendria, Tumera ulmifolia, Nicodemia diversifolia, Tapeinochilos spectabilis, Rauwolfia tetraphylla, Ficus elastica, Cycas circinalis, Caryota urens, Cynnamomum zeylonicum, Aechmealuddemanniana, Phoenix zeylonica, Ficus benjamina, Ficuspumila, Murraya exotica, Trevesia sundaica, Clerodendrumspeciosissimum, Actinidia kolomikta, Paeonia lactifiora, Paeonia suffruticosa, Quercus imbricaria, Iris pallida, Portulacca olleracea, Polygonum aviculare, Iris pseudocarpus, Ailium nutans, Ailium fistulosum, Anthericum ramosum, Veratrum nigrum, Polygonumlapathifolium, Hosta lancifolia, Hosta sieboldii, Echinops sphaerocephalus, Paeonia dahurica, Inula helenium, Crambe pontica, Digitalis lutea, Baptisia australis, Aristolochia australis, Hyssopusserayschanicus, Teucrium chamaedrys, Sedum album, Heracleum pubes cens, Origanum vulgare, Cachrys alpina, Laser trilobum, Matteuccia struthiopteris, Sedum telephium, Bocconia cordata, Ajuga reptans, Thalictrum minus, Anemone japonica, Clematis rectae, Alchemilla officinalis, Potentilla alba, Poterium sangiusorba, Menispermum dauricum, Oxybaphusnyctagineus, Armoracia rusticana, Crambe cordifolia, Agrimoniaeupatoria, Anchusa officinalis, Polemoniumcaeruleum, Valeriana officinalis, Pulmonaria molissima, Stachys lanata, Coronilla varia, Platycarya grandiflora, Lavandula officinalis, Vincetoxicum officinale, Acalypha hispida, Gnetum gnemon, Psychotria nigropunctata, Psychotria metbac teriodomasica, Codiaeum variegatum, Phyllanthus grandifolius, Pterigota alata, Pachyra affinis, Sterculia data, Philodendron speciosum, Pithecellobium unguis-cati, Sanchezia nobilis, Oreopanax capitatus, Ficus triangularis, Kigeliapinnata, Pipercubeba, Laurus nobilis, Erythrina caifra, Metrosideros excelsa, Osmanthus fragrans, Cupres sussempervirens, Jacobinia sp., Senecio platyphylloides, Tetraclinis articulata, Eucalyptus rudis, Podocarpus spinulosus, Eriobotrya japonica, Gingko biloba, Rhododendronsp., Thuja occidentalis, Fagopyrum sufruticosum, Geum macrophyllum, Magnolia kobus, Vinca minor Convallaria majalis, Corylus avellana, Berberis sp., Rosa multifiora, Ostrya carpinifolia, Ostrya connogea, Quercus rubra, Liriodendron tulipifera, Sorbus aucuparia, Betula nigra, Castanea saliva; Bergenia crassifolia, Artemisia dracunculus, Ruta graveolens, Quercus nigra, Schisandra chinensis, Betula alba, Sambucus nigra, Gentiana cruciata, Encephalartos horridus, Phlebodium aureum, Microlepia platyphylla, Ceratozamia mexicana, Stenochlaena tenuifolia, Adiantum trapeziforme, Adiantum raddianum, Lygodium japonicum, Pessopteris crassifolia, Asplenium australasicum, Agathis robusta, Osmunda regaus, Osmundastrum claytonianum, Phyllitis scolopendrium, Polystichum braunii, Cyrtomium fortune, Dryopteris flux mas, Equisetum variegatum, Athyrium nipponicum, Athyrium filix-femina, Parthenocissus tricuspidata, Ligusticum vulgare, Chamaecy parispisifera, Rosa canina, Cotinus coggygria, Celtis occidentalis, Picea schrenkiana, Cyclonia oblonga, Ulmus pumila, Euonymus verrucosus, Deutzia scabra, Mespilus germanica, Quercus castaneifolia, Euonymus europea, Securinega sufruticosa, Koelreuteria paniculata, Syring a josikaea, Zelkova carpinifolia, Abies cephalonica, Taxus baccata, Taxus cuspidata, Salix babylonica, Thuja occidentalis, Actinidia colomicta, Mahonia aquifo hum, Aralia mandschurica, Juglans nigra, Euonymus data, Prinsepia sinensis, Forsythia europaea, Sorbocotoneaster pozdnjakovii, Morus alba, Crataegus macrophyllum, Eucommiaulmifolia, Sorbus commixta, Philodendron amu rense, Cornus mas, Kerria japonica, Panotia persia, Jasminum fruticans, Swidasan guinea, Pentaphylloides fruticosa, Sibiraea altaiensis, Cerasus japonica, Kolkwitzia amabilis, Amigdalus nana, Acer mandschurica, Salix Lama risifolia, Amelanchier spicata, Cerasus mahaleb, Prunus cerasifera, Corylus avellana, Acer tataricum, Viburnum opulus, Syring a vulgaris, Fraxinus exelsior, Quercus trojana, Chaenomeles superba, Pinus salinifolia, Berberis vulgaris, Cotoneaster horisontalis, Cotoneaster fangianus, Fagus sylvatica, Pinuspumila, Pinus sylvestris, Berberis thunbergii, Ajuga forrestii, Anisodus acutangulus, Chinchona ledgerina, Valeriana officinalis, Peganumharmala, Chrysanthemum cineraliaefolium, Tagetes patula, Scopolia japonica, Rauwolfia serpentine, Papaver somniferum, Capsicumfrutescens, Fumaria capreolata L., Datura stramonium, Tinospora rumphii, Tripterygium wilfordii, Coptis japonica, Salvia officinalis, Colleus blumei, Catharanthus roseus, Morinda citrofolia, Lithospermumerythrorhizon, Dioscorea deltoidea, Mueune pruriens, Mirabilis Jalapa, Boerhavia diffusa, Camptotheca acuminate, Nothapodytes foetid, Morus nigra, Symphoricarpus albus and Ophiorrhiza pumila and other chlorophyll bearing plants.

It is understood that plant and fungal sources other than the aforementioned plants or fungi can be used as a source of cyclopiazonic acid, cyclopiazonic acid derivative compounds and starting material that can be used to synthesize cyclopiazonic derivative compounds can be obtained from both natural (Van Breemen et al. (1991) J. Agricul. Food Chem., 39: 1452-1456), and commercial sources. For example, the synthesis outlined in Smith et al. (1987) J. Chem. Res. Synopses, 3: 64-65 or Ma et al. (1995) Tetrahedron: Asymmetry, 6: 313-316, are feasible.

### II. Additional Active Agents.

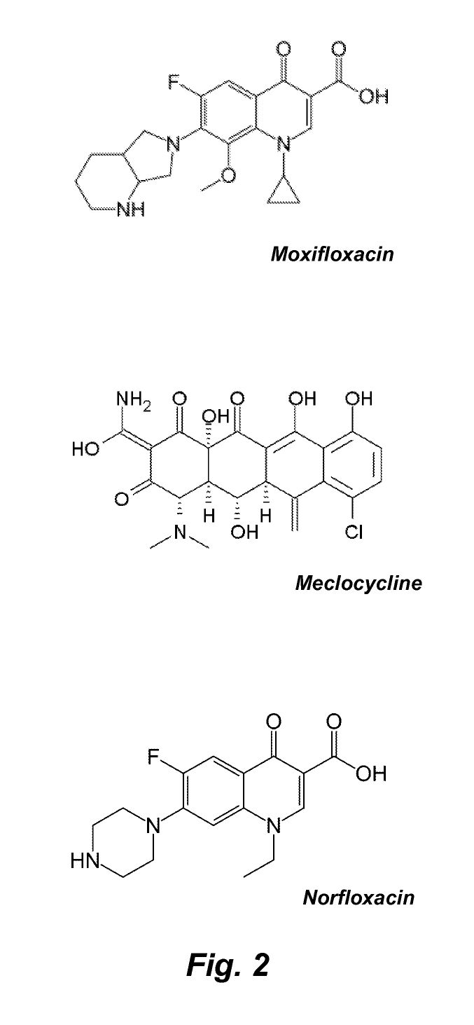

In addition, to cyclopiazonic acid and cyclopiazonic acid derivatives it was also discovered that a number of other agents offer similar radioprotective activities. Such agents include, but are not limited to, minocycline, doxycycline, oxytetracycline, methacycline, rolitetracycline, chlortetracycline, meclocycline, enoxacin, norfloxacin, ciprofloxacin, sarafloxacin, gatifloxacin, levofloxacin, ofloxacin, flumequine, lomefloxacin, moxifloxacin, and 2,5-ditertbutylhydroquinone and/or salts, esters, solvates, or prodrugs thereof. In certain embodiments the agents comprise one or more agents selected from the group consisting of norfloxacin, meclocycline, and moxifloxacin (see, e.g., FIG. 2).

It is contemplated that one or more of these agents can be formulated and used in a manner analogous to the cyclopiazonic acid and cyclopiazonic acid derivatives.

### III. Pharmaceutical Formulation and Administration.

Pharmaceutical Formulations.

In certain embodiments one or more active agents described herein (e.g., cyclopiazonic acid (CPA), cyclopiazonic acid derivative(s), and/or other radioprotective agents described herein) are administered to a mammal in need thereof, e.g., to a mammal exposed to radiation in a clinical or nonclinical setting, or prophylactically in a mammal expected to be exposed to radiation in a clinical or non-clinical setting to prevent or reduce the radiation damage, particularly to otherwise healthy cells and tissues.

The active agent(s) can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience. For example, PCT Publication No: WO 2000/059863 teaches the formulation of disodium salts, monohydrates, and ethanol solvates of a variety of delivery agents.

Similarly, acid salts of active agents (e.g., the therapeutic and/or prophylactic agents described herein) can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of Esters Typically Involves Functionalization of Hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agent(s) identified herein can be administered in a number of ways depending upon whether local or systemic treatment is desired and upon the area to be treated. The agent(s) are useful for parenteral, topical (including ophthalmic), to mucus membranes (including vaginal and rectal delivery), pulmonary (e.g. by inhalation or insufflation of powders or aerosols, including by nebulizer), intratracheal, intranasal, epidermal, transdermal, oral, nasal, subcutaneous, intramuscular, intravenous, or local administration, such as by, for prophylactic and/or therapeutic treatment to exposure or anticipated exposure to radiation and/or in the course of cancer therapy.

The active agents described herein (e.g., cyclopiazonic acid (CPA), cyclopiazonic acid derivative(s), other radioprotective agents described herein) also be combined with a pharmaceutically acceptable carrier and/or excipient to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., cyclopiazonic acid (CPA), cyclopiazonic acid derivative(s), and/or other radioprotective agents described herein,) and the resulting composition is compressed. Where necessary the compressed product is coated, e.g., for masking the taste or for enteric dissolution or sustained release. Suitable coating materials include, but are not limited to, ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets, capsules, gelcaps, and the like, sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising one or more active agent(s) (e.g., cyclopiazonic acid (CPA), cyclopiazonic acid derivative(s), and/or other radioprotective agents described herein) herein can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

For topical administration the active agent(s) described herein can be formulated as solutions, gels, ointments, creams, suspensions, and the like as are well-known in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can optionally contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal or other transepithelial administrations, penetrants appropriate to the barrier to be permeated can be used in the formulation. Such penetrants are generally known in the art.

For oral administration, the compounds can be readily formulated by combining the active agent(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the active agent(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver one or more active agent(s) described herein. Certain organic solvents such as dimethylsulfoxide also can be employed, although usually at the cost of greater toxicity. Additionally, the compounds may be delivered using a sustained-release system, such as semipermeable matrices of solid polymers containing the therapeutic/prophylactic agent(s). Various uses of sustained-release materials have been established and are well known by those skilled in the art. Sustained-release capsules may, depending on their chemical nature, release the compounds for a few days to a few weeks to up to over 100 days. Depending on the chemical nature and the biological stability of the active agent(s), additional strategies for stabilization may be employed.

In certain embodiments, the active agent(s) described herein are administered to the oral cavity. This is readily accomplished by the use of lozenges, aersol sprays, mouthwash, coated swabs, and the like.

In certain embodiments, the active agent(s) of this invention are administered topically, e.g., to the skin surface, to a surgical site, and the like.

In certain embodiments the active agents of this invention are administered systemically (e.g., orally, or as an injectable) in accordance with standard methods well known to those of skill in the art. In other embodiments, the agents, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active agent(s)” that is ultimately available for delivery to the surface of the skin. Thus, for example, the “reservoir” may include the active agent(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

As indicated above, various buccal, and sublingual formulations are also contemplated.

In certain embodiments, one or more active agents of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While pharmacological formulation and administration is described with respect to use in humans, it is also suitable for animal, e.g., veterinary use. Thus certain preferred organisms include, but are not limited to humans, non-human primates, canines, equines, felines, porcines, ungulates, lagomorphs, and the like.

The foregoing formulations and administration methods are intended to be illustrative and not limiting. It will be appreciated that, using the teaching provided herein, other suitable formulations and modes of administration can be readily devised.

In one illustrative embodiment, for preparing a suitable pharmaceutical composition, the active agent(s) are provided as a pure or substantially pure (e.g., greater than 90% pure, preferably greater than about 95% pure, more preferably greater than about 98% or 99% pure and most preferably greater than about 99.9% pure) powder. The pure or substantially pure pure powder composition comprising the active agent(s) is dissolved in CHCl₃ to make a concentration spanning about 1 mg/mL to about 10 mg/mL in a sterile vessel. To this, a detergent (e.g., TWEEN-80) is added in sufficient amounts to make a 1-10% (v/v) detergent concentration in the final sample. The solution is typically homogenous and may be clear green in color. If multiple samples are to be prepared, the solution can be allocated to multiple vessels (e.g., test tubes) at this time. The mixture is then dried under nitrogen, argon, or other suitable gas to dryness. To this dried mixture is added the appropriate amount of water, buffer, or saline solution (sterile) to ¼ to ½ the final volume to be used in treatment. The preparation is then immediately agitated (e.g., sonicated) under warm (60° C. or less) or cold conditions for 1-30 min as needed. Once a clear homogenous solution is reached, the appropriate amount of sterile water, buffer, or saline solution is added to make the final volume required with the detergent (e.g., TWEEN-80) concentration within, but not restricted to 1-10% (v/v) as needed. The preparation is then agitated (e.g., sonicated) another 1-10 min and placed in storage till used. The final preparation is homogenous and clear in consistency.

In an illustrative embodiment, a pharmaceutical composition for oral administration to a mammalian subject is provided, comprising: a) at least one cyclopiazonic acid, cyclopiazonic acid derivative or other radioprotective agent described herein as active ingredient; and b) a vehicle comprising a carrier (e.g., a detergent such as TWEEN-80 (no less than 1%)), and an appropriate bio-compatible solvent such as sterile saline or phosphate buffered saline, etc.

In certain embodiments other suitable carriers include but are not limited to vitamin E TPGS (d-α-tocopheryl polyethylene glycol 1000 succinate, Eastman Chemical Co., Kingsport Term.); saturated polyglycolyzed glycerides such as GELUCIRE™ and LABRASOL™products (Gattefossé Corp., Westwood, N.J.) which include glycerides of C₈-C₁₈ fatty acids; CREMOPHOR™ EL or RH40 modified castor oils (BASF, Mt. Olive, N.J.); MYRJ™ polyoxyethylated stearate esters (ICI Americas, Charlotte, N.C.); TWEEN™ (ICI Americas) and CRILLETT™ (Croda Inc., Parsippany, N.J.) polyoxyethylated sorbitan esters; BRIJ™ polyoxyethylated fatty ethers (ICI Americas); CROVOL™ modified (polyethylene glycol) almond and corn oil glycerides (Croda Inc.); EMSORB™ sorbitan diisostearate esters (Henkel Corp., Ambler, Pa.); SOLUTOL™ polyoxyethylated hydroxystearates (BASF); and β-cyclodextrin.

It will be noted that several of the materials identified as carriers have also been found to be effective co-solubilizers, either alone or in combination with other viscosity-reducing agents, for certain other carriers. In general, any solvent in which cyclopiazonic acid, cyclopiazonic acid derivatives, and/or other radioprotective agents described herein are at least moderately soluble at body temperature or with gentle heating can be used as a co-solubilizer in the vehicle of the novel compositions.

In certain embodiments viscosity-reducing co-solubilizers contemplated for use include, e.g., PHARMASOLVE™ (N-methyl-2-pyrrolidone, International Specialty Products, Wayne, N.J.); MIGLYOL™ glycerol or propylene glycol esters of caprylic and capric acids (HMIs AG, Marl, Germany); polyoxyethylated hydroxystearates (e.g., SOLUTOL™ HS 15); TWEEN™ polyoxyethylated sorbitan esters; SOFTIGEN™ polyethylene glycol esters of caprylic and capric acids (Hütls AG); modified castor oils (such as CREMOPHOR™ EL or RH 40); vegetable oils such as olive oil, sesame oil, polyoxyethylated fatty ethers or modified castor oils; certain saturated polyglycolyzed glycerides (such as a LABRASOL™) citrate esters such as tributyl citrate, triethyl citrate and acetyl triethyl citrate; propylene glycol, alone or in combination with PHARMASOLVE™; ethanol; water; and lower molecular weight polyethylene glycols such as PEG 200 and 400.

The concentration of the active agent(s) in the composition may vary based on the solubility of the active agent in the carrier(s) or carrier(s)/co-solubilizer(s) system and on the desired total dose of active agent(s) to be administered to the patient. In certain embodiments the concentration of cyclopiazonic derivative compound may range from about 0.1, about 1, or about 2 to about 500, about 200, or about 100 mg/ml or mg/g of vehicle, and preferably from about 2 mg/ml to about 50 mg/ml or mg/g.

Other suitable carriers may include mixtures of physiological saline with detergents, e.g., TRITON X-1008 with solvents, such as dimethylsulfoxide (DMSO), or within liposomes. In all cases, any substance used in formulating a pharmaceutical preparation of the invention should be virus-free, pharmaceutically pure and substantially non-toxic in the amount used. One or more penetration enhancers surfactants and chelators may be included. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusid-ate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. Further penetration enhancers include polyoxyethylene-9-lauryl ether, polyoxyethylene-20-cetyl ether.

In another illustrative embodiment suitable for oral administration of the active agent(s), tablets comprising the active agent(s) combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia are provided. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the cyclopiazonic acid, cyclopiazonic acid derivatives, and/or other radioprotective agent(s) described herein can be combined with various sweetening or flavoring agents, coloring matter or dyes, and, if so desired, emulsifying and/or suspending agents as well, together with such diluents as water, ethanol, propylene glycol, glycerin and various like combinations thereof.

Prodrugs.

In certain embodiments prodrug and/or extended release formulations of the radioprotective agents described herein are contemplated.

In certain embodiments prodrug and/or extended release formulations of the radioprotective agents described herein are contemplated. It will be recognized that a rapid-onset and a steady level of a radioprotective agent is preferred for effective radioprotection. Prodrug and extended/controlled release formulations can be used to provide such a dosage regime.

In certain embodiments, the use of polymeric drug deliver system sis contemplated. Controlled drug delivery occurs when a polymer, whether natural or synthetic, is combined with the active agent( ) in such a way that the active agent(s) are released from the material in a predesigned manner. The release of the active agent may be constant over a long period, it may be cyclic over a long period, or it may be triggered by the environment or other external events. IN particular the sue of controlled-delivery systems can result in the maintenance of drug levels within a desired range, the need for fewer administrations, optimal use of the drug in question, and increased patient compliance.

A wide range of materials have been employed to control the release of drugs and other active agents and the use of these materials with the radioprotectve agnts described herein is contemplated. Some suitable materials include but are not limited to poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, and polyorthoesters. There are three primary mechanisms by which active agents can be released from a delivery system: diffusion, degradation, and swelling followed by diffusion. Any or all of these mechanisms may occur in a given release system. Diffusion occurs when a drug or other active agent passes through the polymer that forms the controlled-release device. The diffusion can occur on a macroscopic scale—as through pores in the polymer matrix—or on a molecular level, by passing between polymer chains.

Other polymeric delivery system are known to those of skill in the art. For example, U.S. Pat. No. 5,942,252 describes a microcapsule comprising as its biocompatible excipient a poly(lactide-co-glycolide), poly(lactide), poly(glycolide), copolyoxalate, polycaprolactone, poly(lactide-co-caprolactone), poly(esteramide), polyorthoester, poly(p-hydroxybutyric) acid and/or polyanhydride for use in delivering agents into and through mucosally-associated lymphoid tissue.

PCT Publication WO 98/36013 describes aliphatic-aromatic dihydroxy compounds for use as controlled drug delivery systems. PCT Publication WO 97/39738 describes preparation of microparticles of a sustained release ionic conjugate comprising a free carboxyl group containing biodegradable polymers and a free amino group-containing drug. PCT Publication WO 02/09768 discloses [polymers (i.e. polyesters, polyamides, and polythioesters or a mixture thereof) that comprise active agent(s) and degrade hydrolytically into the biologically active agents.

In certain embodiments the use of nanoparticle foformulatiosn is contemplated. For drug delivery not only engineered particles may be used as carrier, but also the drug itself may be formulated at a nanoscale, and then function as its own “carrier”. The composition of the engineered nanoparticles may vary. Source materials may be of biological origin like phospholipids, lipids, lactic acid, dextran, chitosan, or have more “chemical” characteristics like various polymers (e.g., the polymers described above), carbon, silica, and metals.

Other suitable prodrug formulations include, for example, the use of amino, or otherwise modified, derivatives of the active agents described herein. IN this regard, it is noted that U.S. Patent publication No: 20060287283 teaches prodrugs of 9-aminomethyltetracycline compounds and it is contemplated that the active agents described herein can be similarly modified.

Effective Dosages

The active agents described herein (e.g., cyclopiazonic acid (CPA), cyclopiazonic acid derivative(s), and/or other radioprotective agents described herein) will generally be used in an amount effective to achieve the intended purpose (e.g., to reduce, repair, or prevent radiation-induced damage to cells, tissues, or organs). Of course, it is to be understood that the amount used will depend on the particular application. By therapeutically effective amount is meant an amount of active agent or composition comprising such that inhibits or eliminates the progression of radiation-induced damage to cells, tissues, or organs or that aids in the reversal of radiation induced damage to cells, tissues, or organs. By prophylactically effective amount is meant an amount of active agent or composition comprising such that prevents or inhibits the progression of radiation-induced damage to cells, tissues or organs when they are exposed to radiation after administration of the radioprotective agent(s). An ordinarily skilled artisan will be able to determine effective amounts of particular active agent(s) or combinations thereof for particular applications without undue experimentation using, for example, in vitro or in vivo assays known to those of skill in the art.

In certain therapeutic applications, the compositions of this invention are administered, e.g., topically administered or administered to the oral or nasal cavity, or to a mucosa (e.g., vaginal, pulmonary, rectal, etc.) to a subject suffering from radiation exposure (clinical or non-clinical) or at risk for radiation exposure prophylactically to prevent or reduce radiation-induced damage.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compositions of the present invention, and the delivery means, and can generally be estimated based on EC₅₀'s found to be effective in in vitro and in vivo animal models.

The dosage/amount of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 150 mg/kg/day. In certain embodiments, dosages range from about 20 mg to about 100 mg given orally twice daily. It will be appreciated that such dosages may be varied to optimize a therapeutic and/or phophylactic regimen in a particular subject or group of subjects. Determination of a therapeutically effective amount is well within the capabilities of those skilled in the art, especially in light of the detailed disclosure provided herein.

As in the case of disinfectants and preservatives, for topical administration to treat or prevent bacterial, yeast, fungal or other infections a therapeutically effective dose can be determined using, for example, the in vitro assays provided in the examples. The treatment may be applied while the infection is visible, or even when it is not visible. An ordinarily skilled artisan will be able to determine therapeutically effective amounts to treat topical infections without undue experimentation.

For systemic administration, a therapeutically effective dose can be estimated initially from in vitro assays. For example, a dose can be formulated in animal models to achieve a circulating cyclic peptide concentration range that includes the IC₅₀ as determined in cell culture (i.e., the concentration of test compound that is lethal to 50% of a cell culture), the MIC, as determined in cell culture (i.e., the minimal inhibitory concentration for growth) or the IC₁₀₀ as determined in cell culture (i.e., the concentration of peptide that is lethal to 100% of a cell culture). Such information can be used to more accurately determine useful doses in humans.

Initial dosages can also be estimated from in vivo data, e.g., animal models, using techniques that are well known in the art. One having ordinary skill in the art could readily optimize administration to humans based on animal data. In certain embodiments dosage amount and interval can be adjusted individually to provide plasma levels of the active agent(s) that are sufficient to maintain therapeutic or prophylactic effect.

In cases of local administration or selective uptake, the effective local concentration of active agent(s) may not be related to plasma concentration. One having skill in the art will be able to optimize therapeutically effective local dosages without undue experimentation.

Toxicity

Preferably, a therapeutically effective dose of the cyclopiazonic acid (CPA), cyclopiazonic acid derivative(s), and/or other radioprotective agents described herein described herein will provide therapeutic benefit without causing substantial toxicity.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds that exhibit high therapeutic indices are preferred, particularly for in vivo applications. The data obtained from cell culture assays and animal studies can be used in formulating a dosage range that is not toxic for use in human. The dosage of the peptides described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (see, e.g., Fingl et al. (1975) In: The Pharmacological Basis of Therapeutics, Ch.1, p. 1).

IV. Use in Conjunction with Antineoplastic Agents.

In certain embodiments methods are contemplated comprising the use of one or more radioprotective agents described herein in combination with one or more antineoplastic (anti-cancer) agents. In certain embodiments combined formulastiosn are contemplated comprising a combination of one or more radioprotective agents described herein and one or more antineoplastic (anti-cancer) agents.

Various classes of antineoplastic (e.g., anticancer) agents are contemplated for use such embodiments. Such anticancer agents include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like. Additional other cytotoxic, chemotherapeutic or anti-cancer agents contemplated for use include alkylating agents or agents with an alkylating action, such as cyclophosphamide (CTX; e.g. cytoxan®); anti-metabolites, such as methotrexate (MIX) and 5-fluorouracil (5-FU); antibiotics; other antitumor agents, such as paclitaxel and pactitaxel derivatives, the cytostatic agents, glucocorticoids and corticosteroids such as prednisone, leucovorin, folinic acid and other folic acid derivatives, and similar, diverse antitumor agents.

Partiuclar illustrative suitable anti-cancer agents in the methods and combined formulastion described herein include, but are not limited to Such agents include, but are not limited to alkylating agents (e.g., mechlorethamine (Mustargen), cyclophosphamide (Cytoxan, Neosar), ifosfamide (Ifex), phenylalanine mustard; melphalen (Alkeran), chlorambucol (Leukeran), uracil mustard, estramustine (Emcyt), thiotepa (Thioplex), busulfan (Myerlan), lomustine (CeeNU), carmustine (BiCNU, BCNU), streptozocin (Zanosar), dacarbazine (DTIC-Dome), cis-platinum, cisplatin (Platinol, Platinol AQ), carboplatin (Paraplatin), altretamine (Hexylen), etc.), antimetabolites (e.g. methotrexate (Amethopterin, Folex, Mexate, Rheumatrex), 5-fluoruracil (Adrucil, Efudex, Fluoroplex), floxuridine, 5-fluorodeoxyuridine (FUDR), capecitabine (Xeloda), fludarabine: (Fludara), cytosine arabinoside (Cytaribine, Cytosar, ARA-C), 6-mercaptopurine (Purinethol), 6-thioguanine (Thioguanine), gemcitabine (Gemzar), cladribine (Leustatin), deoxycoformycin; pentostatin (Nipent), etc.), antibiotics (e.g. doxorubicin (Adriamycin, Rubex, Doxil, Daunoxome-liposomal preparation), daunorubicin (Daunomycin, Cerubidine), idarubicin (Idamycin), valrubicin (Valstar), mitoxantrone (Novantrone), dactinomycin (Actinomycin D, Cosmegen), mithramycin, plicamycin (Mithracin), mitomycin C (Mutamycin), bleomycin (Blenoxane), procarbazine (Matulane), etc.), mitotic inhibitors (e.g. paclitaxel (Taxol), docetaxel (Taxotere), vinblatine sulfate (Velban, Velsar, VLB), vincristine sulfate (Oncovin, Vincasar PFS, Vincrex), vinorelbine sulfate (Navelbine), etc.), chromatin function inhibitors (e.g., topotecan (Camptosar), irinotecan (Hycamtin), etoposide (VP-16, VePesid, Toposar), teniposide (VM-26, Vumon), etc.), hormones and hormone inhibitors (e.g. diethylstilbesterol (Stilbesterol, Stilphostrol), estradiol, estrogen, esterified estrogens (Estratab, Menest), estramustine (Emcyt), tamoxifen (Nolvadex), toremifene (Fareston) anastrozole (Arimidex), letrozole (Femara), 17-OH-progesterone, medroxyprogesterone, megestrol acetate (Megace), goserelin (Zoladex), leuprolide (Leupron), testosteraone, methyltestosterone, fluoxmesterone (Android-F, Halotestin), flutamide (Eulexin), bicalutamide (Casodex), nilutamide (Nilandron), etc.) inhibitors of synthesis (e.g., aminoglutethimide (Cytadren), ketoconazole (Nizoral), etc.), immunomodulators (e.g., rituximab (Rituxan), trastuzumab (Herceptin), denileukin diftitox (Ontak), levamisole (Ergamisol), bacillus Calmette-Guerin, BCG (TheraCys, TICE BCG), interferon alpha-2a, alpha 2b (Roferon-A, Intron A), interleukin-2, aldesleukin (ProLeukin), etc.) and other agents such as 1-aspariginase (Elspar, Kidrolase), pegaspasgase (Oncaspar), hydroxyurea (Hydrea, Doxia), leucovorin (Wellcovorin), mitotane (Lysodren), porfimer (Photofrin), tretinoin (Veasnoid), and the like.

### V. Kits.

In another embodiment this invention provides kits for the inhibition of an infection and/or for the treatment and/or prevention of dental caries in a mammal and/or the inhibition of biofilms (e.g., on a prosthetic or medical implant). The kits typically comprise a container containing one or more of the active agents, e.g., cyclopiazonic acid (CPA), cyclopiazonic acid derivative(s), and/or other radioprotective agents described herein. In certain embodiments the active agent(s) can be provided in a unit dosage formulation (e.g., suppository, tablet, caplet, patch, etc.) and/or may be optionally combined with one or more pharmaceutically acceptable carriers and/or excipients.

In addition, the kits optionally include labeling and/or instructional materials providing directions (i.e., protocols) for the practice of the methods or use of the “therapeutics” or “prophylactics” of this invention. Preferred instructional materials describe the use of one or more active agent(s) of this invention therapeutically or prophylactically to inhibit or prevent damage to cells, tissues, or organs from exposure to radiation. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

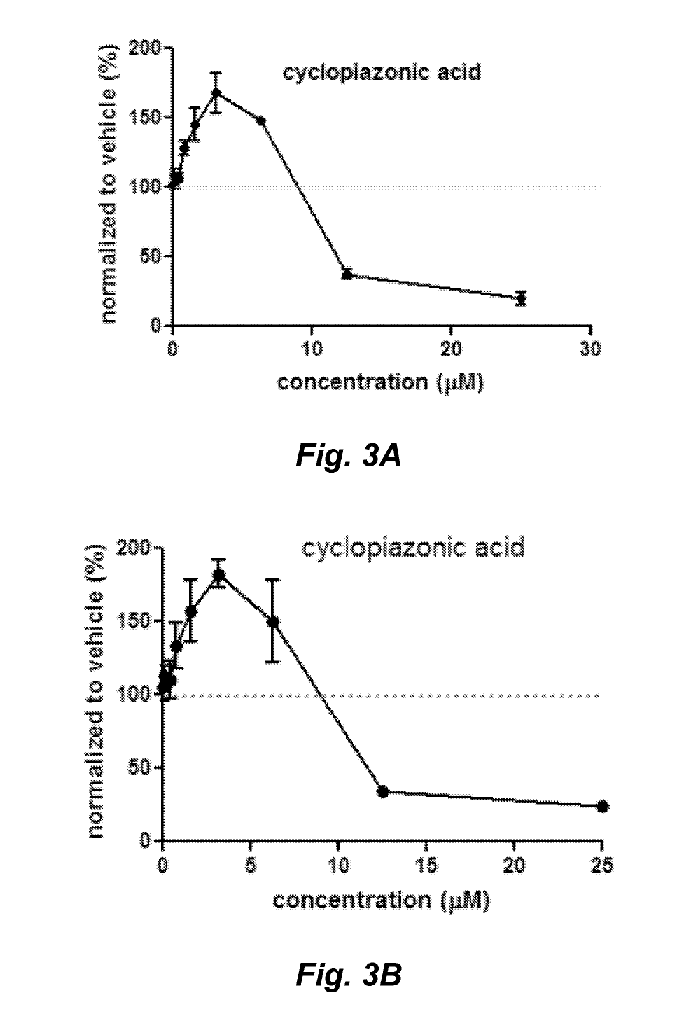

Dose-responses of irradiated cells treated with cyclopiazonic acid (CPA) were determined. A cell viability assay with TiL1 cells was measured using ATPlite reagent 24 hours after irradiation with 2Gy. CPA was added to the cells 3 h before irradiation for protection (FIG. 3A), or 1 hour after irradiation mitigation (FIG. 3B) activities. The percent cell viability plotted was normalized to vehicle control value. As shown in FIGS. 3A and 3B CPA both protects and mitigates TiL1 cell from radiation damage.

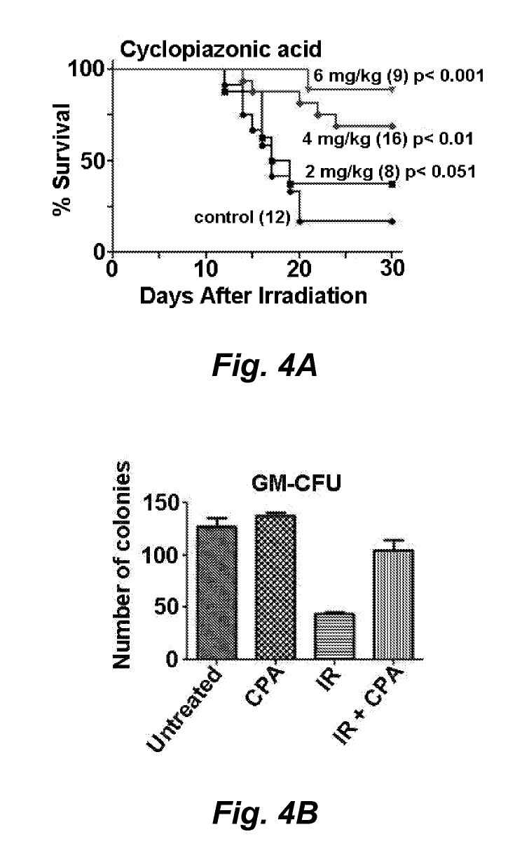

The effect of CPA on animal survival against a lethal dose total body irradiation was determined. Two oral administrations of CPA at 24 h and 1 h prior to irradiation at 8 Gy protected mice from radiation-induced death (FIG. 4A). This effect was most prominent with CPA treatment at 6 mg/kg showing 89% survival while only 17% of controls survived.

CPA at 6 mg/kg or vehicle control was administered twice prior to irradiation as described above along with un-irradiated control mice for Granulocyte-macrophage colony forming units. Bone marrow cells were collected from 4 mice per each treatment group 3 d after total body irradiation and Gm-CFU was counted 8-9 d after plating bone marrow cells. * indicates p<0.05 for IR vs IR+CPA comparison, showing that CPA protects the immunohematopoietic system from a lethal dose total body irradiation (FIG. 4B).

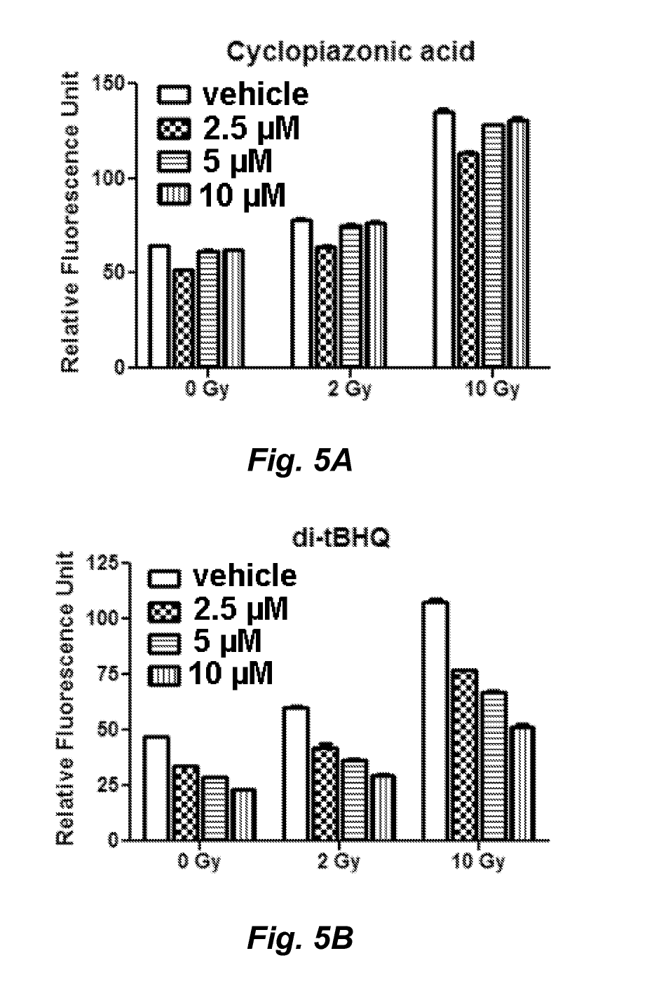

The effect of CPA on ROS scavenging was also evaluated. CPA did not reduce the irradiation induced reactive oxygen species (FIG. 5A), while di-tBHQ did in dose-responsive manner (FIG. 5B). The intracellular ROS was measured immediately after irradiation in TiL-1 cells using 2′,7′-dichlorofluorescein diacetate (DCF-DA, Invitrogen). The compound and DCF-DA probe at 25 μM was added 3 h and 1 h before irradiation, respectively.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for protecting a cells or tissues in a subject from radiation damage, or reducing radiation damage to cells or tissues in a subject, said method comprising administering to the subject cyclopiazonic acid (CPA) and/or a cyclopiazonic acid derivative in an amount effective to reduce radiation damage in a cell or tissue in said subject.

2. The method of claim 1, wherein said cyclopiazonic acid (CPA) and/or a cyclopiazonic acid derivative comprises a compound according the formula: 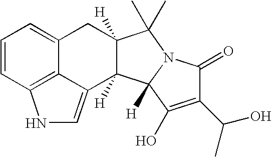
or a pharmacologically acceptable salt, or solvate thereof.

3. The method of claim 1, wherein said cyclopiazonic acid (CPA) and/or a cyclopiazonic acid derivative comprises a compound according to the formula: 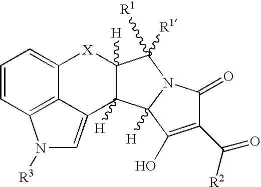
wherein
X is selected from the group consisting of CH₂, O, NH, C₂H₄ and S;
R¹ and R^{1′} are independently selected from the group consisting of H, F, Cl, CH₃, CH₂OH, and NH₂;
R² is selected from the group consisting CH₃, (CH₂)ₙCH₃ where n=1, 2, 3 or 4, OH, (CH₂)ₙOH where n=1, 2, 3 or 4, NH₂, ester linked and ether linked alkyl group of the formula (CH₂)ₙCH₃ where n is between 0 and 24 and contains 0, 1, 2, 3 double bonds and 0, 1, 2, or 3 hydroxy moieties and one or two carbonyl moieties; and
R³ is selected from the group consisting of H, methyl, ethyl, propyl, iso-propyl, butyl, iso-butyl, CF₃, CCl₃, benzyl and substituted benzyl derivatives, anthranyl and substituted derivatives, tosyl/sulfonamide, and an amino acid.

4. The method of claim 3, wherein R¹ and R¹′ respectively are selected from the group consisting of H and H, H and Cl, H and F, F and F, CH₃ and H, CH₂OH and H, NH₂ and H, and CH₂OH and CH₃.

5. The of claim 2, wherein R² comprises a moiety selected from the group consisting of a CH₂, a CH₃, an H, an OH, a hemisuccinate, a choline, a phosphate, a phosphoryloxymethylcarbonyl, an amino acid, a dimethylaminoacetate, a phosphonate, an N-alkoxycarbonyl, and a phosphoryloxymethyloxycarbonyl.

6. The method of claim 2, wherein R² and/or R³ comprise an amino acid selected from the group consisting of alanine, arginine, asparagine, aspartic acid, cysteine, glutamine, glutamic acid, glycine, histidine, isoleucine, leucine, isoleucine, lysine, methionine, phenylalanine, proline, pyrrolysine, serine, selenocysteine, threonine, tryptophan, tyrosine, and valine.

7. The method of claim 2, wherein said cyclopiazonic acid (CPA) and/or a cyclopiazonic acid derivative comprises a compound according the formula:

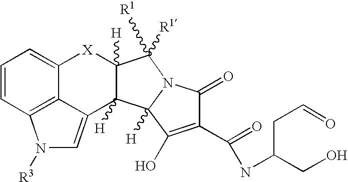

8. The method of claim 7, wherein X is CH₂, or CAL.

9. The of claim 7, wherein R¹ and R¹′ respectively are selected from the group consisting of H and H, H and Cl, H and F, F and F, CH₃ and H, CH₂OH and H, NH₂ and H, and CH₂OH and CH₃.

10. The method of claim 7, wherein R³ is H.

11. The method of claim 2, wherein said cyclopiazonic acid (CPA) and/or a cyclopiazonic acid derivative comprises a compound according the formula:

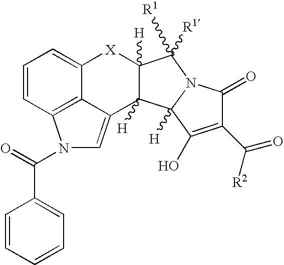

12. The method of claim 11, wherein X is CH₂, or C₂H₄.

13. The method of claim 11, wherein R¹ and R¹′ respectively are selected from the group consisting of H and H, H and Cl, H and F, F and F, CH₃ and H, CH₂OH and H, NH₂ and H, and CH₂OH and CH₃.

14. The method of claim 11, wherein R² is H.

15. The method of claim 1, wherein said cyclopiazonic acid (CPA) and/or a cyclopiazonic acid derivative is administered before exposure of said subject to radiation.

16. The method of claim 1, wherein said cyclopiazonic acid (CPA) and/or a cyclopiazonic acid derivative is administered during exposure of said subject to radiation.

17. The method of claim 1, wherein said cyclopiazonic acid (CPA) and/or a cyclopiazonic acid derivative is administered after exposure of said subject to radiation.

18. The method of claim 1, wherein said cyclopiazonic acid (CPA) and/or a cyclopiazonic acid derivative is combined with a pharmaceutically acceptable excipient or carrier.

19. The method of claim 18, wherein said excipient or carrier is formulated to provide sustained release of said cyclopiazonic acid (CPA) and/or a cyclopiazonic acid derivative for a period of at least 8 hours.

20. The method of claim 18, wherein said excipient or carrier is formulated for administration via a route selected from the group consisting of oral administration, inhalation, rectal administration, surgical implantation, transdermal administration, parenteral administration, intravenous administration, subcutaneous administration, and topical administration.

21. The method of claim 1, wherein cyclopiazonic acid (CPA) and/or a cyclopiazonic acid derivative is administered via a route selected from the group consisting of oral administration, inhalation, rectal administration, surgical implantation, transdermal administration, parenteral administration, intravenous administration, subcutaneous administration, and topical administration.

22. The method of claim 1, wherein said cells or tissues comprise a hematopoietic tissue or a mucosal tissue.

23. The method of claim 1, wherein said subject is a non-human mammal.

24. The method of claim 1, wherein said subject is a human.

25. The method of claim 1, wherein said radiation is produced in a therapeutic treatment.

26. The method of claim 25, wherein said radiation is produced by an implanted radiation source and/or by a beam radiation source.

27. (canceled)

28. The method of claim 1, wherein said cyclopiazonic acid (CPA) and/or a cyclopiazonic acid derivative is administered in conjunction with an anti-cancer drug.

29. The method of claim 1, wherein said radiation is produced in a non-clinical setting.

30. A method of cancer radiotherapy or radiosurgery, said method comprising: administering to non-tumor cells and/or tissues in a subject in need of such therapy an amount of a cyclopiazonic acid (CPA) and/or a cyclopiazonic acid derivative effective to reduce radiation damage to the non-tumor cells and tissues; and subjecting a tumor or a metastatic cell in said subject to radiation.

31. The method of claim 30, wherein the tumor or metastatic cell to be treated is of a cancer selected from the group consisting of lung cancer, colorectal cancer, NSCLC, bronchoalveolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis.

32. The method of claim 31, wherein the tumor or tumor metastasis is refractory.

33. The method of claim 30, wherein said cyclopiazonic acid (CPA) and/or a cyclopiazonic acid derivative comprises a compound according the formula: 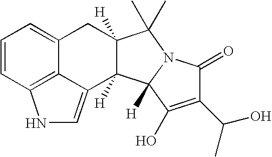
or a pharmacologically acceptable salt, or solvate thereof.

34. The method of claim 30, wherein said cyclopiazonic acid (CPA) and/or a cyclopiazonic acid derivative comprises a compound according the formula: 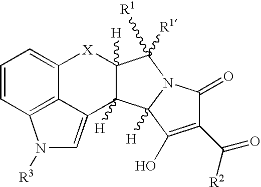
wherein
X is selected from the group consisting of CH₂, O, NH, C₂H₄ and S;
R¹ and R^{1′} are independently selected from the group consisting of H, F, Cl, CH₃, CH₂OH, and NH₂;
R² is selected from the group consisting CH₃, (CH₂)ₙCH₃ where n=1, 2, 3 or 4, OH, (CH₂)₁₀H where n=1, 2, 3 or 4, NH₂, ester linked and ether linked alkyl group of the formula (CH₂)ₙCH₃ where n is between 0 and 24 and contains 0, 1, 2, 3 double bonds and 0, 1, 2, or 3 hydroxy moieties and one or two carbonyl moieties; and
R³ is selected from the group consisting of H, methyl, ethyl, propyl, iso-propyl, butyl, iso-butyl, CF₃, CCl₃, benzyl and substituted benzyl derivatives, anthranyl and substituted derivatives, tosyl/sulfonamide, and an amino acid.

35-37. (canceled)

38. The method according to claim 34, wherein said cyclopiazonic acid (CPA) and/or a cyclopiazonic acid derivative comprises a compound according to the formula:

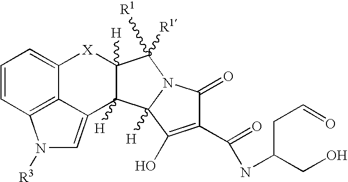

39. The method of claim 38, wherein X is CH₂, or C₂H₄.

40-41. (canceled)

42. The method according to claim 34, wherein said cyclopiazonic acid (CPA) and/or a cyclopiazonic acid derivative comprises a compound according the formula:

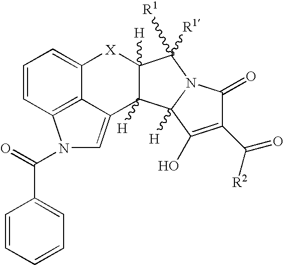

43-45. (canceled)

46. The method according to any one of claims 30-45, wherein said cyclopiazonic acid (CPA) and/or a cyclopiazonic acid derivative is administered before or during exposure of said subject to radiation.

47-52. (canceled)

53. The method of claim 30, wherein said cells or tissues comprise a hematopoietic tissue or a mucosal tissue.

54. (canceled)

55. The method of claim 30, wherein said subject is a human.

56. The method of claim 30, wherein said radiation is produced by an implanted radiation source or by a beam radiation source.

57. (canceled)

58. The method of claim 30, wherein said cyclopiazonic acid (CPA) and/or a cyclopiazonic acid derivative is administered in conjunction with an anti-cancer drug.

59. A method of protecting biological material from radiation damage, or reducing radiation damage in biological material, said method comprising exposing the biological material to a cyclopiazonic acid (CPA) and/or a cyclopiazonic acid derivative in an amount sufficient to reduce or inhibit damage from exposure to radiation.

60-61. (canceled)

62. A pharmaceutical composition comprising cyclopiazonic acid (CPA) and/or a cyclopiazonic acid derivative in a pharmaceutically acceptable excipient or carrier.

63. The composition of claim 62, wherein said cyclopiazonic acid (CPA) and/or a cyclopiazonic acid derivative comprises a compound according the formula: 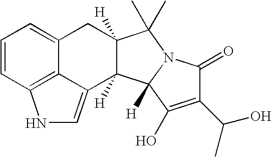
or a pharmacologically acceptable salt, or solvate thereof.

64. The composition of claim 62, wherein said cyclopiazonic acid (CPA) and/or a cyclopiazonic acid derivative comprises a compound according to the formula: 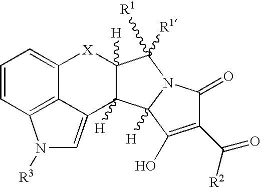
wherein
X is selected from the group consisting of CH₂, O, NH, C₂H₄ and S;
R¹ and R^{1′} are independently selected from the group consisting of H, F, Cl, CH₃, CH₂OH, and NH₂;
R² is selected from the group consisting CH₃, (CH₂)ₙCH₃ where n=1, 2, 3 or 4, OH, (CH₂)₁₀H where n=1, 2, 3 or 4, NH₂, ester linked and ether linked alkyl group of the formula (CH₂)ₙCH₃ where n is between 0 and 24 and contains 0, 1, 2, 3 double bonds and 0, 1, 2, or 3 hydroxy moieties and one or two carbonyl moieties; and
R³ is selected from the group consisting of H, methyl, ethyl, propyl, iso-propyl, butyl, iso-butyl, CF₃, CCl₃, benzyl and substituted benzyl derivatives, anthranyl and substituted derivatives, tosyl/sulfonamide, and an amino acid.

65-67. (canceled)

68. The composition according to claim 64, wherein said cyclopiazonic acid (CPA) and/or a cyclopiazonic acid derivative comprises a compound according the formula:

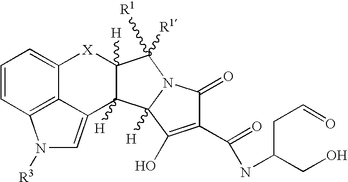

69-71. (canceled)

72. The composition according to claim 64, wherein said cyclopiazonic acid (CPA) and/or a cyclopiazonic acid derivative comprises a compound according the formula:

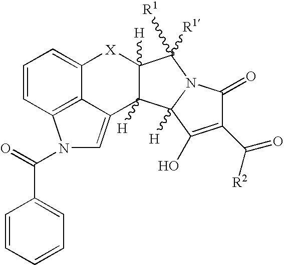

73-75. (canceled)

76. The composition of claim 62, wherein said excipient or carrier is for administration in a modality suitable for inhibiting cell or tissue damage from radiation exposure.

77. The composition of claim 62, additionally comprising one or more other anti-cancer agents.

78. The composition of claim 77, wherein said other anti-cancer agent is selected from the group consisting of an alkylating drug, an antimetabolite, a microtubule inhibitor, a podophyllotoxin, an antibiotic, a nitrosourea, a hormone, a kinase inhibitor, an activator of tumor cell apoptosis, and an antiangiogenic agent.

79. A pharmaceutical composition for oral administration to a mammalian subject, comprising:
a) cyclopiazonic acid (CPA) and/or a cyclopiazonic acid derivative according to Formula II as active ingredient; and
b) a vehicle comprising:
i) a TWEEN surfactant at ranging from 0.01% to about 10% by volume in a biologically compatible solvent; and
ii) a carrier comprising at least 1-30% Vitamin E TPGS.

80-81. (canceled)

82. A method for treating tumors or tumor metastases in a patient, comprising: administering to said patient a therapeutically effective amount of a pharmaceutical composition comprising at least one cyclopiazonic acid (CPA) and/or a cyclopiazonic acid derivative according to Formula II in pharmaceutically acceptable excipient, carrier or vehicle.

83. (canceled)

84. The method of claim 82, wherein the tumor or tumor metastases to be treated is selected from the group consisting of lung cancer, colorectal cancer, NSCLC, bronchoalveolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors and tumor metastases

85-86. (canceled)

87. The method of claim 82, additionally comprising administering one or more other anti-cancer agents.

88. (canceled)

89. The method of claim 82, wherein said composition is administered to prevent and/or treat non-cancer diseases or conditions that result from changes in cellular proliferation selected from benign hypertrophy of tissues, arthritis, retinal ailments, skin abnormalities, scar formation, cardiovascular diseases, gastrointestinal dysfunction, hematologic illness, immunological imbalance, allergies, gynecological and urological problems.

90. The method of claim 82, wherein said composition is administered to prevent and/or treat non-cancer diseases or conditions that result from changes in angiogenesis process selected from ailments/conditions that result from too high or too low levels of blood vessel formation.

91. The method of claim 82, wherein said composition is administered to treat one or more infections caused by one or multiple agents selected from bacteria, fungi, viruses, mycobacteria, and yeast as a consequence of radiation exposure.

92. A method for protecting a cell and/or a tissue, and/or an organ in a subject from radiation damage, or reducing radiation damage to cells or tissues in a subject, said method comprising administering to the subject an agent selected from the group consisting of norfloxacin, meclocycline, and moxifloxacin in an amount effective to reduce radiation damage in a cell, tissue, or organ in said subject.
